(19) United States
(12) Patent Application Publication
(10) Pub. No.: US 2022/0257969 A1
(43) Pub. Date: Aug. 18, 2022

(54) METHOD FOR TREATING PELVIC PAIN, CHRONIC PROSTATITIS, AND OR OVERACTIVE BLADDER SYMPTOMS

(71) Applicant: RALPH ZIPPER, MELBOURNE, FL (US)

(72) Inventor: RALPH ZIPPER, MELBOURNE, FL (US)

(21) Appl. No.: 17/732,435

(22) Filed: Apr. 28, 2022

Related U.S. Application Data
(63) Continuation of application No. 15/452,958, filed on Mar. 8, 2017, now abandoned.
Application No. 15/452,958 is a continuation-in-part of application No. 12/687,991, filed on Jan. 15, 2010, now Pat. No. 10,743,929.
Application No. 12/687,991 is a continuation-in-part of application No. 12/496,216, filed on Jul. 1, 2009, now Pat. No. 8,795,264.
Application No. 15/452,958 is a continuation-in-part of application No. 12/687,965, filed on Jan. 15, 2010, now abandoned.
Application No. 12/687,965 is a continuation-in-part of application No. 12/496,216, filed on Jul. 1, 2009, now Pat. No. 8,795,264.
(60) Provisional application No. 61/077,348, filed on Jul. 1, 2008.
(60) Provisional application No. 61/077,348, filed on Jul. 1, 2008.

(51) Int. Cl. A61N 5/06; A61B 18/20; A61B 18/14; A61B 18/24

(52) U.S. Cl. CPC A61N 5/0603; A61B 18/20; A61B 18/1402; A61B 2018/00964; A61N 5/0601; A61N 5/06; A61B 2018/2005; A61B 18/24

(57) ABSTRACT

A method for treating pelvic pain and or chronic prostatitis, and or overactive bladder symptoms. Energy, preferably in the form of infrared or near infrared wavelength light may be applied across the vaginal tissue or rectal tissue to treat pelvic pain and or chronic prostatitis. This method of energy application may cause local heating, alteration of cellular respiration, and alterations of local blood flow resulting in decreased muscle spasm, and or decreased pain, and or decreased overactive bladder symptoms. The method of the present invention may effectively treat chronic pelvic pain and or the symptoms of chronic prostatitis. The method of the invention may combine massaging of tissue with irradiation of same.

### CROSS REFERENCE TO RELATED APPLICATIONS AND INCORPORATION BY REFERENCE

This non provisional patent application filed under 35 U.S.C. 111(a) is a continuation of U.S. patent application Ser. No. 15/452,958, entitled METHOD FOR TREATING PELVIC PAIN, CHRONIC PROSTATITIS, AND OR OVERACTIVE BLADDER SYMPTOMS, filed in the United States Patent and Trademark Office (USPTO) on Mar. 8, 2017, which published from the USPTO as U.S. Patent Application Publication No. US 2017-0172658 A1 on Jun. 22, 2017, which is incorporated herein by reference in its entirety; Ser. No. 15/452,958 is a continuation in part of U.S. patent application Ser. No. 12/687,991, entitled Bulbous Tipped Surgical Device and Method for Decreasing the Size and/or Changing the Shape of Pelvic Tissues, which was filed in the USPTO on Jan. 15, 2010, which published from the USPTO as U.S. Patent Application Publication No. US 2011-0004202 A1 on Jan. 6, 2011, and which issued as U.S. Pat. No. 10,743,929 on Aug. 18, 2020, which is incorporated herein by reference in its entirety; Ser. No. 12/687,991 was a continuation in part of U.S. patent application Ser. No. 12/496,216, entitled METHOD FOR DECREASING THE SIZE AND/OR CHANGING THE SHAPE OF PELVIC TISSUES, filed in the USPTO on Jul. 1, 2009, which published from the USPTO as U.S. Patent Application Publication No. US 2010-0004644 A1 on Jan. 7, 2010, and which issued as U.S. Pat. No. 8,795,264 on Aug. 5, 2014, which is also incorporated herein by reference in its entirety; Ser. No. 12/496,216 was a non provisional of provisional patent application Ser. No. 61/077,348, entitled Method for Decreasing the Size and/or Changing the Shape of the Vagina, Labia, Prepuce, Perineum and/or Surrounding Supportive Tissues, filed with the USPTO on Jul. 1, 2008, which is incorporated by reference herein in its entirety; Ser. No. 15/452,958 is also a continuation in part of U.S. patent application Ser. No. 12/687,965, entitled Laser Device and Method for Decreasing the Size and/or Changing the Shape of Pelvic Tissues, filed in the USPTO on Jan. 15, 2010, which published from the USPTO as U.S. Patent Application Publication No. US 2011-0004203 A1 on Jan. 6, 2011, which is incorporated by reference herein in its entirety; Ser. No. 12/687,965 was a continuation in part of U.S. patent application Ser. No. 12/496,216, entitled METHOD FOR DECREASING THE SIZE AND/OR CHANGING THE SHAPE OF PELVIC TISSUES, filed in the USPTO on Jul. 1, 2009, which published from the USPTO as U.S. Patent Application Publication No. US 2010-0004644 A1 on Jan. 7, 2010, and which issued as U.S. Pat. No. 8,795,264 on Aug. 5, 2014, which is also incorporated herein by reference in its entirety; Ser. No. 12/496,216 was a non provisional of provisional patent application Ser. No. 61/077,348, entitled Method for Decreasing the Size and/or Changing the Shape of the Vagina, Labia, Prepuce, Perineum and/or Surrounding Supportive Tissues, filed with the USPTO on Jul. 1, 2008, which is incorporated by reference herein in its entirety. Each of the foregoing U.S Patent Application Publications and U.S. patents are incorporated herein by reference in their entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

### 1. Field of the Invention

The present invention generally relates to a method of applying laser energy as a treatment regimen to the vagina or rectum. More specifically, the present invention relates to a method of transmitting laser energy across vaginal and or rectal tissue to treat the causes and symptoms of chronic pelvic pain, chronic prostatitis, and overactive bladder.

### 2. Background Art

Pelvic disorders such as chronic pelvic pain and chronic prostatitis are highly prevalent and highly debilitating. Over 50 million U.S. women and over 100 million women worldwide suffer from chronic pelvic pain. In the absence of readily available and effective treatments, only 40% of chronic pelvic pain patients are referred to specialists. Over 8 percent of men suffer from chronic prostatitis. This is a prevalence similar to diabetes and heart disease. Similar to chronic pelvic pain, there are few effective treatments. Up to 90% of men with chronic prostatitis meet the criteria for chronic nonbacterial prostatitis/chronic pelvic pain syndrome, a disorder with few treatment options supported by only limited evidence. Although approximately 16% of men and women suffer from the symptoms of overactive Bladder, multiple treatments are available. Existing treatments for overactive Bladder include prescription medications taken in perpetuity, with numerous side effects, repetitive injections of botulinum toxin with associated side effects and only transient improvement, implantable neuromodulation devices costing fifteen to twenty thousand dollars per implant, and repetitive percutaneous nerve stimulation. There are no non-systemic, non-invasive treatments for overactive bladder symptoms.

What is needed in the art, therefore, is a non-invasive method for treating the above identified conditions that is safe and effective.

### BRIEF SUMMARY OF THE INVENTION

The present invention comprises an apparatus and method that have one or more of the following features and/or steps, which alone or in any combination may comprise patentable subject matter.

The present invention overcomes the shortcomings of the prior art in that it provides a non-invasive method for treating the above identified conditions that is safe and effective.

In accordance with one embodiment, a method for treating pelvic pain and or chronic prostatitis, and or overactive bladder symptoms by way of the trans-tissue transmission of laser energy, preferably in the infrared or near infrared spectrum, wherein the method comprises the steps of providing a probe capable of emitting said energy to and through the vaginal and or rectal tissue, inserting said probe into the vagina and or rectum, and activating the source of the energy. In the preferred embodiment, the probe is thence kept in continuous, back and forth, motion until the appropriate dose of energy has been administered.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 depicts an exemplary flow chart for the first embodiment of the method of the invention for treating the vagina of a patient.

FIG. 2 depicts a laser source and probe system such as may be utilized in carrying out the steps of the invention.

FIG. 3A depicts a probe apparatus such as may be utilized in carrying out the steps of the invention, depicting a probe with a bulbous or spherical massaging light emitting portion and depicting an exemplary radiation pattern. FIG. 3B depicts an exemplary cross section of the probe depicted in FIG. 3A.

FIGS. 4A and 4B depicts a conical embodiment of probe apparatus such as may be utilized in carrying out the steps of the invention.

FIGS. 5A and 5B depicts a trapezoidal embodiment of probe apparatus such as may be utilized in carrying out the steps of the invention.

FIG. 6 depicts a generalized probe apparatus such as may be utilized in carrying out the steps of the invention for use in treating vaginal tissue, depicting energy radiating from the probe in a direction along a longitudinal axis of the probe in a spherical or partially spherical pattern.

FIG. 7 depicts a probe apparatus such as may be utilized in carrying out the steps of the invention, depicting the probe inserted just beyond the opening of the vagina of a patient, and showing laser energy emitted from the probe. FIG. 6 Further depicts a probe apparatus such as may be utilized in carrying out the steps of the invention, depicting the probe inserted into the vagina of a patient, depicting the vaginal tissue conforming to the bulbous tip of the probe, and depicting the total distance of travel L of the laser probe within the vagina for an embodiment of the method of the invention.

FIGS. 8A and 8B depict a generalized probe apparatus such as may be utilized in carrying out the steps of the invention for use in treating the prostate, depicting energy radiating from a side of the probe in a direction transverse to a longitudinal axis of the probe so as to illuminate the length of the tissue overlying the prostate. FIG. 8A depicts a side view, and FIG. 8B depicts an end view.

FIG. 9 depicts a probe apparatus such as may be utilized in carrying out the steps of the invention for use in treating the prostate, depicting the probe in relation to the rectum walls, and depicting the rectum walls conforming around the bulbous tip of the probe. FIG. 9 further depicts the probe inserted into the rectum at the point to the point where the first (proximal) mark is located at the anus, at which point the laser energy source may be activated and deactivated. FIG. 9 also depicts and further depicts the probe inserted into the rectum to the point where the second (distal) mark is located at the anus marks on the probe.

FIG. 10 depicts an exemplary pulsatile waveform of an embodiment of the method of the invention.

FIG. 11 depicts an embodiment of a probe of the invention, showing embodiment of a temperature sensor disposed on the probe for measuring the temperature of body tissue.

FIG. 12a depicts an embodiment of a probe of the invention for vaginal use, in which the massaging portion and the light emitting portion are separated by a distance Z.

FIG. 12b depicts an embodiment of a probe of the invention for rectal use, in which the massaging portion and the light emitting portion are separated by a distance Z.

FIG. 13 depicts a block diagram of an embodiment of the temperature probe and laser source controller of the invention.

In the figures, like callout numbers refer to like elements.

### DETAILED DESCRIPTION OF THE INVENTION

The following documentation provides a detailed description of the invention.

Although a detailed description as provided in the attachments contains many specifics for the purposes of illustration, anyone of ordinary skill in the art will appreciate that many variations and alterations to the following details are within the scope of the invention. Accordingly, the following preferred embodiments of the invention are set forth without any loss of generality to, and without imposing limitations upon, the claimed invention. Thus the scope of the invention should be determined by the appended claims and their legal equivalents, and not merely by the preferred examples or embodiments given.

As used herein, “memory”, “medium”, “media”, “computer readable memory”, “computer readable medium”, “storage media”, “computer readable storage media” and “computer readable storage medium” shall include within their meanings only physical non-transitory computer readable hardware, and such terms shall specifically exclude signals per se, carrier waves, propagating signals and other transitory signals. Such physical non transitory computer readable media may comprise hardware memory that comprises a physical structure for storing data which may include computer executable instructions or data.

As used herein, “controller” includes within its meaning any electrical device or combination of electrical devices capable of executing computer readable instructions such as a controller, processor, microcontroller, microprocessor, field programmable gate array, programmable logic array, embedded firmware, virtual machine, combinational logic or any other electrical or electronic device or any combination of devices known in the electrical arts as capable of executing computer readable or hardcoded instructions.

As used herein, with respect to the controller, “adapted to” includes within its meaning that the controller executes computer readable non-transitory or hardcoded instructions, or utilizes hardwired logic or any other means known in the art for controlling a feature, device, or signal, to carry out the subject function. Any function herein described as being caused or commanded by the controller of the invention may be carried out by the controller executing computer readable non-transitory instructions, or utilizing hardwired logic, programmable logic such as gate arrays, or any other means known in the art for controlling a feature, device, or signal, to carry out the subject function

As used herein, “near infrared” means between 700 nm and 1,400 nm.

Power and energy density measurements are referenced to the surface of the probe light emitting portion 101 (or 101a, 101b, 101c, etc.). In the case which the power or energy density is not uniform across the surface that is emitting the laser energy, the power or energy density given is an average power density as measured across the total surface area emitting the laser energy.

The scope and breadth of the present inventive disclosure is applicable across a wide variety of procedures, tissues and anatomical structures. Although the following detailed description contains many specifics for the purposes of illustration, anyone of ordinary skill in the art will appreciate that many variations and alterations to the following details are within the scope of the invention. The method of the invention may be applied to any tissue, and thus is not limited to rectal or vaginal tissue only. Accordingly, the following preferred embodiments of the invention are set forth without any loss of generality to, and without imposing limitations upon, the claimed invention.

Each of the preferred embodiments of the treatment method of the invention may share the clinically effective novel dosing regimen and parameters of the invention that were discovered and developed only through the extensive experimentation by the inventor, including multiple clinical trials. The experimentation led to a determination of the specific claimed treatment parameters that were observed to be efficacious when laser energy was applied to body tissue using a probe in the manner described herein. These novel dosing and treatment parameters comprise the administration of light energy at of 4-10 watts for vaginal use and 2-8 watts for rectal use; the administration of between 2000 and 4000 joules of near infrared light per treatment; the administration of such energy at a power density between 0.09 W/cm² and 6.0 W/cm² measured at the exterior surface of the light emitting portion of the laser probe; and the movement of the probe along the treatment area at a rate between 2.5 millimeters per second and 60 millimeters per second to provide both a massaging and an irradiating effect. Ideally, the power density (W/cm²) to probe speed ratio shall (mm/sec) was experimentally determined to be between 0.0042 and 1.8. Outside this range, therapeutic effect was lost.

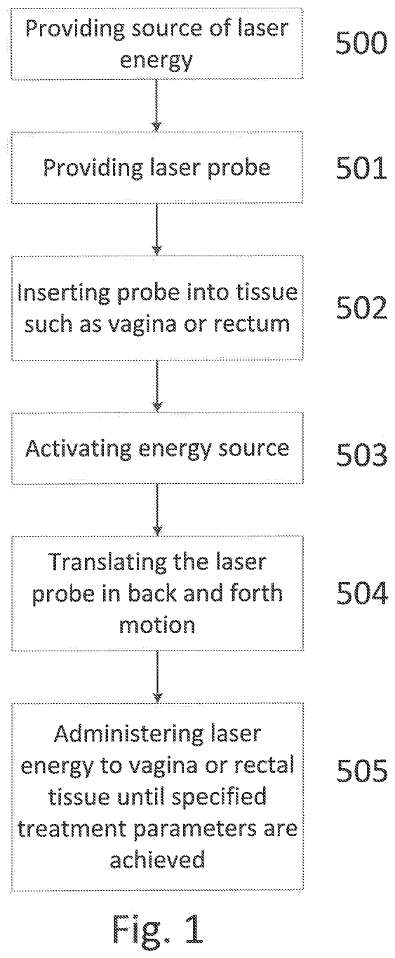

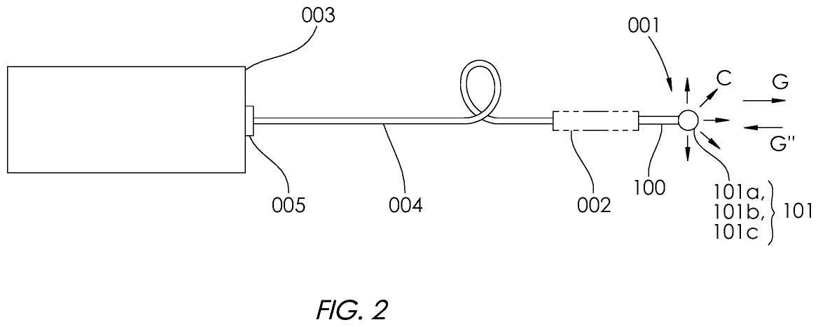

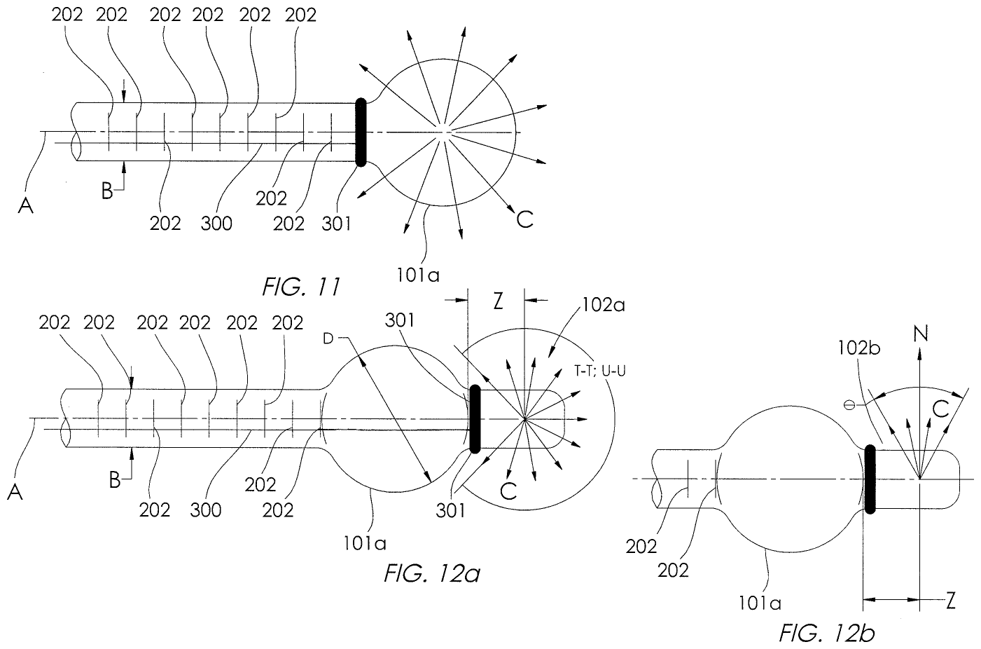

Referring now to FIGS. 1 and 2, the method of the invention comprises, generally, the steps of providing a source of laser energy 500, providing a probe 001 capable of emitting laser energy to body tissue such as vaginal tissue or rectal tissue, probe 001 being in optical communication with said source of laser energy 003 via optical fiber or cable 004, and probe body portion 100 which attaches probe portion 101 to probe handle 002. Handle 002 may be any apparatus that is adapted to advance or retract, or both, probe body portion 100 in the direction of arrows G and G′ which are oriented along an axis of the probe. Likewise, although specific embodiments of probe 001 are described herein, probe 001, which comprises a probe body portion 100 and a probe massaging portion 101, may be any probe capable of emitting laser energy at near infrared wavelengths. In an embodiment, probe massaging portion 101 may be the portion of the probe that emits light energy. In other embodiments in which the probe massaging portion 101 and the portion of the probe that emits light energy are different features, it is preferred, but not necessary, that these features be located along the probe within one centimeter of each other so that the illumination of body tissue with near infrared light from the light emitting portion of the probe is simultaneously, or nearly simultaneously, receiving a massaging effect by the translation of the probe massaging portion 101 along the treated tissue. In other embodiments, the light emitting portion is defined as being a separate structure from the massaging portion such as depicted in FIGS. 12A and 12B.

Still referring to FIG. 1, the method further comprises the step of inserting the probe into body tissue such as the vagina or rectum of a person, who may be a patient receiving treatment, 502, such that at least a portion of the probe is in physical contact with a portion of said body tissue, followed by the step of activating the source of laser energy 503. The probe may then be translated according various embodiments of the invention to provide a massaging effect on the vaginal or rectal tissue 504, or until a specific treatment parameter has been reached, 505, or both.

Referring now to FIG. 2, a laser source and probe system such as may be utilized in carrying out the steps of the invention is depicted.

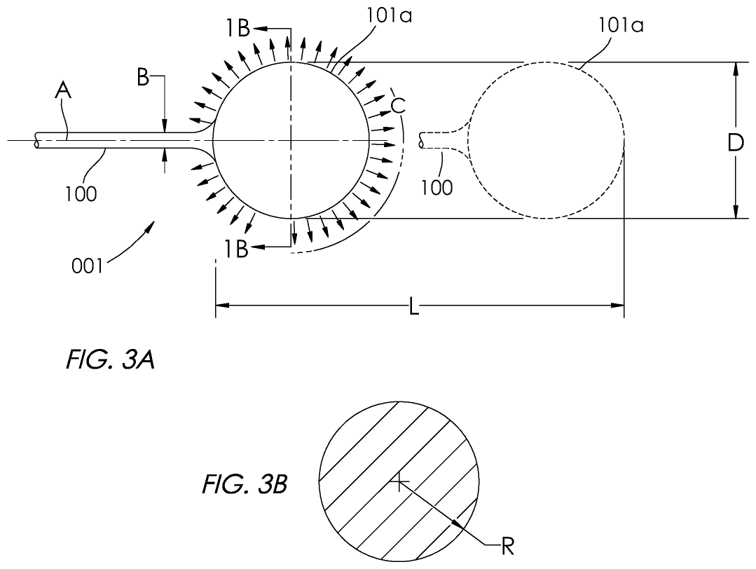

The probe may, by way of example and not by limitation, be laser probes such as the exemplary embodiments of probes depicted in FIGS. 3A, 3B, 4A, 4B, 5A and 5B. While these exemplary shapes are depicted, it is understood that the scope of the invention includes any shape for which a massaging portion 101 of the probe is of greater outer dimension than probe body portion 100.

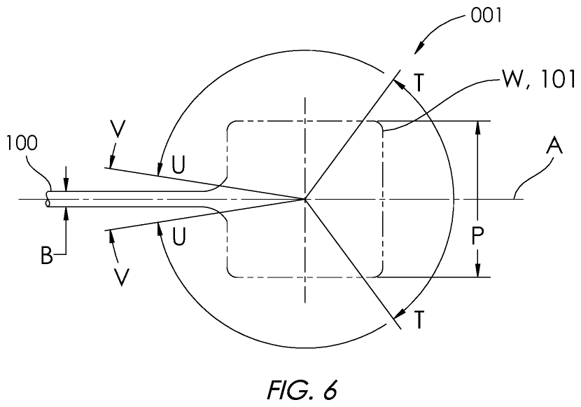

Referring now to FIGS. 3A and 3B, a spherical embodiment of a probe 001 that may be used to carry out the method of the invention is depicted. Probe body portion 100 has outer dimension B and communicates laser energy from laser source 003 (not shown in FIGS. 3A and 3B, but depicted in FIG. 2) to probe light emitting portion 101, which is, in the embodiment depicted, a spherical or bulbous shape of radius R. During use in a method of the invention, probe 001 may be translated a distance L, measured as depicted in the figure. Light energy C may be projected outwards from massaging portion 101 in a substantially or partially spherical pattern, except for the area of the sphere attached to probe connector 100 which may be of outer dimension B as is further discussed in relation to FIG. 6 below.

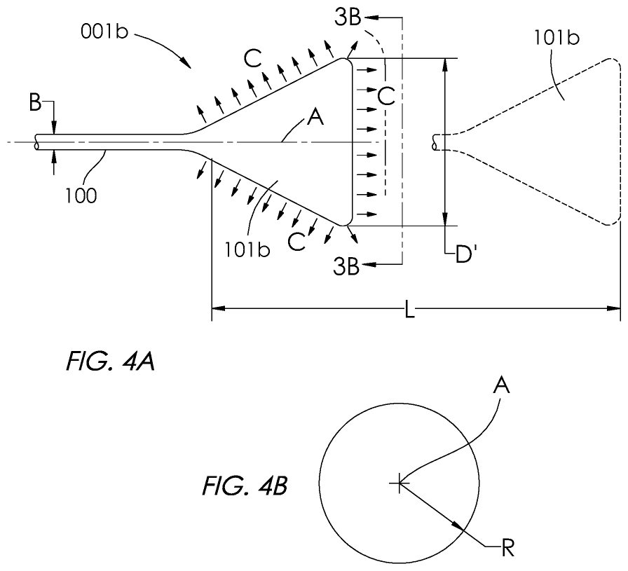

Referring now to FIGS. 4A and 4B, a conical embodiment of a probe 001 that may be used to carry out the method of the invention is depicted. Probe body portion 100 has outer dimension B and communicates laser energy from laser source 003 (not shown in FIGS. 4A and 4B, but depicted in FIG. 2) to probe light emitting portion 101, which is, in the embodiment depicted, a conical shape having an end face of radius R. During use in a method of the invention, probe 001 may be translated a distance L, measured as depicted in the figure. Light energy C may be projected outwards from massaging portion 101, except for the area of the massaging portion attached to probe body portion 100 which may be of outer dimension B.

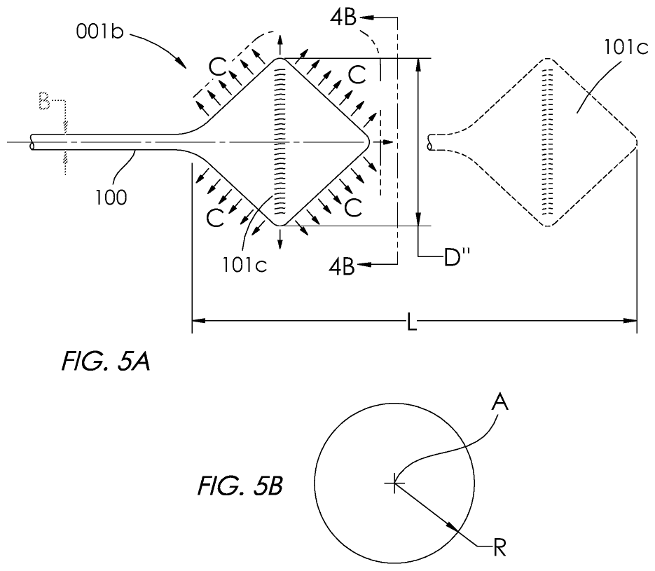

Referring now to FIGS. 5A and 5B, an embodiment of a probe 001 that may be used to carry out the method of the invention is depicted. Probe body portion 100 has outer dimension B and communicates laser energy from laser source 003 (not shown in FIGS. 5A and 5B, but depicted in FIG. 2) to probe light emitting portion 101, which, in the embodiment depicted, has an outer dimension characterized by radius R. During use in a method of the invention, probe 001 may be translated a distance L, measured as depicted in the figure. Light energy C may be projected outwards from massaging portion 101, except for the area of the massaging portion attached to probe body portion 100 which may be of outer dimension B.

In the embodiments depicted in FIGS. 3A, 3B, 4A, 4B, 5A, and 5B, the light emitting portion and the massaging portion are the same structure 101a, b, or c respectively.

Referring now to FIG. 6, a probe 001 having any outer shape W such as may be utilized in carrying out the steps of the invention for use in treating vaginal tissue, depicting energy radiating in a partially spherical pattern from the probe in a direction along a longitudinal axis of the probe A in a radiation pattern is depicted. In FIG. 6, outer shape W of probe massaging portion 101 is depicted in broken lines to indicate that the probe massaging portion 101 may be spherical, conical, trapezoidal or any three dimensional shape. While spherical, conical, and trapezoidal shapes are described herein as examples of the shape of the massaging portion 101, it is understood that the scope of the invention includes any shape for which outer dimension P is greater than dimension B, i.e., for which the massaging portion of the probe is of greater outer dimension than the probe body portion 100. In this embodiment of the probe, light is emitted in a radiation pattern that ranges from partially spherical as depicted by angle T-T which may be for example 120°, to nearly spherical as depicted by angle U-U, and having an axis that is coaxial with the longitudinal axis A of the probe. In this context, “nearly spherical” means a radiation pattern that is spherical except for the partially spherical angle depicted as V-V in which little or no light may be transmitted due to probe body portion 100 having an outer dimension B. Thus, the value of partially spherical angle V-V is dependent upon the value of B. The radiation pattern thus described causes the treatment of the vaginal walls encountered during energy transmission without the need for rotation of the probe in order to fully illuminate the treated vaginal walls. In yet another preferred embodiment, the center of the radiation pattern may be along an axis that is transverse to the longitudinal axis of the probe A. By way of example, in a 180 degree or half sphere radiation pattern along such a transverse axis, treatment could be limited to the rectal or bladder side of the vagina and such side could be treated without rotation of the probe.

### Vaginal Treatment

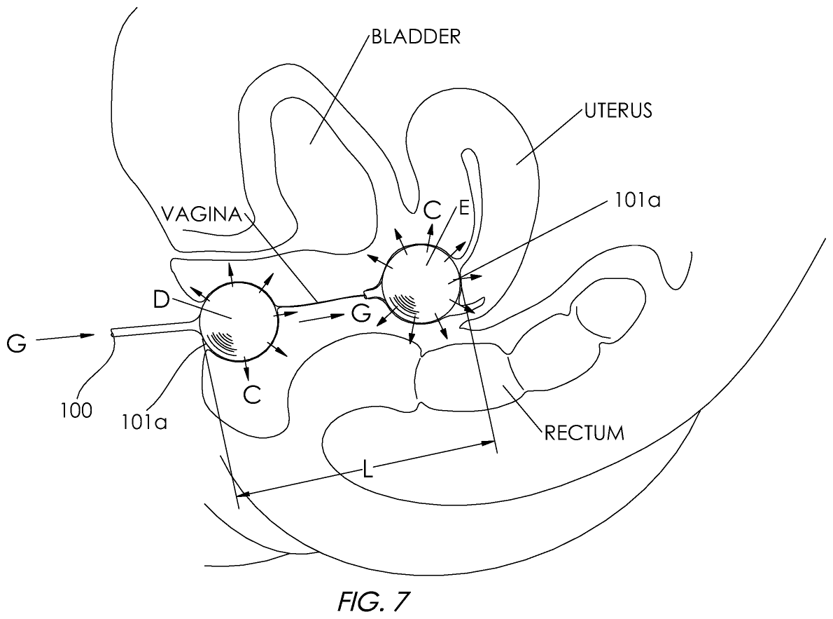

Referring now to FIG. 7, in an embodiment, the method of the invention is directed to treating vaginal tissue. In this embodiment, the laser probe massaging portion 101 (depicted for example as a spherical massaging portion 101a) is inserted into a vagina in the direction of arrow G, the source of laser energy is activated such that either continuous wave or pulsatile laser energy is transmitted from the laser source, through the probe body portion 100, and into the surrounding vaginal tissue thus treating this tissue. The laser probe massaging portion 101 may then may be translated along the probe longitudinal axis A (not shown in FIG. 6 but shown in FIG. 3A), in a back and forth fashion, over a treatment length L between proximal position D and distal position E, until specific experimentally derived treatment parameters for the delivery of laser energy to the vaginal tissue surrounding the laser probe are met. Probe body portion 100 is depicted for reference. Certain anatomical structures, including the bladder, rectum and uterus, are also depicted for reference. Although FIG. 6 depicts a probe comprising a spherical or bulbous massaging portion 101a, any shape probe may be used.

Still referring to FIG. 7, the back and forth motion of the probe over distance L between positions D and E may be, and is preferably, continuous. Continuous is defined as back and forth movement that is interrupted in the instant of a change in direction when the probe has traversed distance L and is located at either proximal position D or distal position E such as is created by an in and out, back and forth, or to and fro movement. This movement of the probe within the vagina creates a mechanical massage effect on the vaginal tissue that is in physical contact with massaging portion 101. Multiple years of clinical investigation has led to the invention of a specific range of treatment parameters. Outside of these treatment parameters, efficacy was lost and or patient discomfort was encountered. These treatment parameters include movement of the probe at a rate between 2.5 millimeters per second and 60 millimeters per second, a total dose of 200-600 joules per linear centimeter of vagina treated or 13.045-310.546 joules/cm², whereas such area is calculated using the linear distance of vagina treated and the radius of the energy emitting portion of the probe, and a power density is between 0.09 W/cm² and 6.0 W/cm². In a preferred embodiment, the diameter of the energy emitting portion of the probe is between 1.5 and 3.5 cm, the probe velocity is between 10 mm/sec and 40 mm/sec, the power density (W/cm²) to probe speed (mm/sec) ratio shall be between 0.0042 and 1.8, total dose is 200-400 joules per linear centimeter or 21.068-149.208 joules/cm2, and the power density between 0.09 W/cm² and 6.0 W/cm². Distance L is defined as the distance between the most proximal and most distal points of treatment. Once the above noted dose has been administered, the energy source is deactivated and the probe is removed from the vagina. If the energy source is capable of recording and or otherwise utilizing the linear distance, such information shall be measured and entered in to the energy source prior to the transmission of laser energy through the probe. With regard to dosing by joules/cm², it is important to understand that the vagina exists as a potential space rather than an open space. Once a probe is placed into the vagina, the potential space is opened, the vaginal tissue conforms around the probe, and the diameter of the space thence becomes the diameter of the inserted probe. This Probe Treatment Surface Area, PTSA, has not been previously described, creates an objective measurement allowing for a reproducible treatment protocol with resultant reproducible safety and efficacy.

In a further embodiment of the method for treating a vagina, the energy is transmitted in a pulsatile or continuous fashion until a total dose of 200-600 joules per linear centimeter or 13.045-310.546 joules/cm² of energy has been transmitted into vaginal tissue, wherein such area is calculated using the linear distance of vagina treated and the radius of the energy emitting portion of the probe, and a power density is between 0.09 W/cm² and 6.0 W/cm². This is the therapeutic range shown to be safe, comfortable, and effective through extensive experimentation. In a preferred embodiment, the diameter of the energy emitting portion of the probe is between 1.5 and 3.5 cm, the power density (W/cm²) to probe speed (mm/sec) ratio is between 0.0042 and 1.8, total dose is 200-400 joules per linear centimeter or 21.068-149.208 joules/cm², and the power density is between 0.09 W/cm2 and 6.0 W/cm². In a preferred embodiment, the probe projects said energy in in a 120-360 degree partially spherical to nearly spherical radiation pattern having an axis that is co-axial with probe longitudinal axis A as depicted in FIG. 6 and described above in relation thereto.

In an embodiment, once the probe is inserted in a vagina, the energy source 003 is activated and laser energy is preferably transmitted though the probe massaging portion 101 or light emitting portion 102a as shown in FIG. 12A and into the vaginal tissue surrounding the probe. Distance L is defined as the distance between the most proximal and most distal points of treatment. The laser energy source 003 is then inactivated and the probe is advanced or withdrawn within the vagina by one centimeter. This advancement or withdrawal may be facilitated by markings on probe body portion 100 that are visible from outside the vagina when the probe is inserted into the vagina. This process is repeated until the vaginal canal or painful area of the pelvic floor is fully treated; i.e., until one of the dosage parameters has been met. The energy source is then deactivated and the probe is removed from the vagina.

The vaginal treatment methods of the invention may be facilitated by a group of novel device features. Such features include markings on the probe connector portion 100 to demonstrate distance of insertion and a temperature sensor on the probe which provides feedback to the energy source. In one embodiment, the energy source adjusts pulse shape and/or amplitude and or time between pulses in order to maintain surface temperature between 40 and 45 degrees centigrade. In another embodiment, the energy source shall adjust pulse shape and/or amplitude and or time between pulses in order to maintain surface below 45 degrees centigrade. Auditory and or visual cues may be provided by the energy source user interface and or other hardware to facilitate the movement of the probe at the prescribed rate. Such ques may rely upon the input of the probe diameter or power density information into the energy source and or an accelerometer contained within the probe or handle of the probe. Such cues may also rely upon surface temperature measurements taken by a temperature measuring mechanism within the probe that provides such temperature measurement to the energy source and or accessory hardware. In the preferred embodiment, the probe shall have a variation in outside dimension of any cross sectional shape such that a massage or milking effect is created on the treated tissue. By way of example and not limitation, this alteration in probe outer dimension may take a spherical shape (see FIGS. 3A and 3B), conical shape (see FIGS. 4A and 4B), or trapezoidal shape (see FIGS. 5A and 5B). The shape of the probe massaging portion outer surface 101 may take any three dimensional shape; the example shapes depicted in FIGS. 3A, 3B, 4A, 4B, 5A, and 5B are exemplary and not exhaustive.

### Prostate Treatment

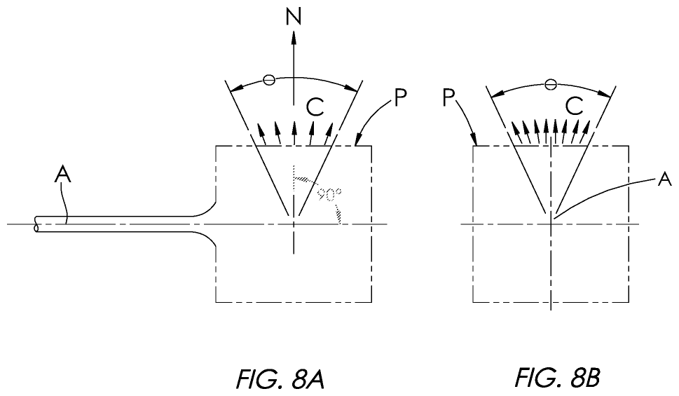

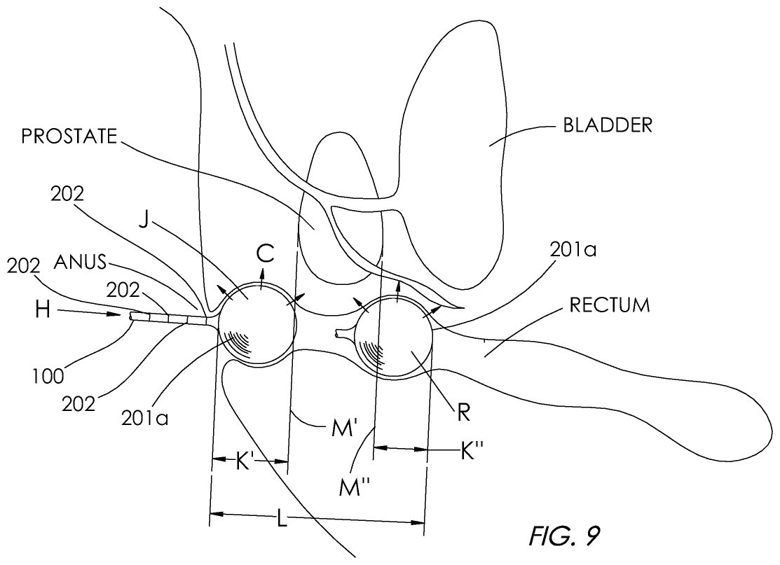

Referring now to FIGS. 8A, 8B, and 9, in a further embodiment, the method of the invention is directed to treating a prostate by irradiating through the rectum wall into the prostate. This embodiment of the invention involves the placement of a probe of the invention attached to an energy source capable of transmitting continuous wave or pulsatile near infrared wavelengths though said probe, into the rectum, by inserting probe massaging portion 201 into the rectum in the direction of arrow G. In a preferred embodiment, the probe is adapted such that it projects said laser energy in a partially spherical radiation pattern having an axis N as shown in FIGS. 8A and 8B in which N is orthogonal to the probe longitudinal axis A, and in which 0 is between 300-180°. Once inserted in the rectum the probe is oriented such that the emitted laser energy is projected predominantly toward the prostate, the energy source is activated and, thence, laser energy is transmitted though the probe and into the surrounding rectal tissue, passing through the rectal tissue and into the prostate. The proper orientation of the probe may be facilitated by a mark on the handle of the probe or a shape of the handle probe that allows the surgeon or other person performing the method of the invention to align the transmitted optical energy from the probe in the direction of the prostate. The probe is then translated in a back and forth motion over a distance L between positions J and R until specified parameters are achieved. This back and forth translation may be performed continuously, with exception for the instant in which direction of movement is reversed. This translation creates a mechanical massage effect. Multiple years of clinical investigation led to the invention of a specific range of treatment parameters. Outside of these treatment parameters, efficacy was lost and or patient discomfort was encountered. Treatment parameters include movement of the probe at a rate between 2.5 millimeters per second and 60 millimeters per second, a total dose of 200-600 joules per linear centimeter of rectum treated or 13.045-310.546 joules/cm², whereas such area is calculated using the linear distance of rectum treated and the radius of the energy emitting portion of the probe, and a power density is between 0.09 W/cm² and 6.0 W/cm². In a preferred embodiment, the diameter of the energy emitting portion of the probe shall be between 1.5 and 3.5 cm, the probe speed shall be between 10 mm/sec and 40 mm/sec, the power density shall be between 0.09 W/cm² and 6.0 W/cm², the power density (W/cm²) to probe speed ratio between 0.0042 and 1.8, total dose shall equal 200-400 joules per linear centimeter or 21.068-149.208 joules/cm², and the power density shall be between 0.09 W/cm² and 6.0 W/cm². The distance L is defined as the distance between the most proximal and most distal points of treatment. Once the above noted dose has been administered, the energy source is deactivated and the probe is removed from the rectum. In the preferred embodiment, the linear distance is that distance beginning one centimeter proximal to the prostate and ending one centimeter distal to the prostate. In the preferred embodiment, markings on the probe body portion 100 are used to identify the range of rectum lying beneath and preferably, but not necessarily slightly beyond the margins of the typical prostate and movement occurs between such markings, the linear distance. In an alternative embodiment, the distance between such markings may correspond to the length of the prostate to be treated. If the energy source is capable of recording and or otherwise utilizing the linear distance, such information shall be measured and entered in to the energy source prior to the transmission of laser energy through the probe.

In an embodiment of the method of the invention directed to treating a prostate by transmitting laser energy in a pulsatile fashion, such energy may emitted by the massaging portion of the probe until a total dose of 200-600 joules per linear centimeter or 13.045-310.546 joules/cm² of energy has been emitted from the probe, wherein such area is calculated using the linear distance of rectum treated and the radius of the energy emitting portion of the probe, and a power density is between 0.09 W/cm² and 6.0 W/cm². This is the therapeutic range shown to be safe through extensive experimentation. In a preferred embodiment, the diameter of the energy emitting portion of the probe shall be between 1.5 and 3.5 cm, the power density (W/cm²) to probe speed (mm/sec) ratio shall be between 0.0042 and 1.8, total dose shall equal 200-400 joules per linear centimeter or 21.068-149.208 joules/cm², and the power density shall be between 0.09 W/cm² and 6.0 W/cm². Distance L is defined as the distance between the most proximal and most distal points of treatment. The energy source is inactivated and the probe is advanced or withdrawn by one centimeter. This advancement or withdrawal may be facilitated by markings 202 on the probe. This process is repeated until the area overlying the prostate has received a predetermined dose of energy. The energy source is deactivated and the probe is removed from the rectum. Ideally, the pulse shape and time as well as the time between pulses shall be set to maintain a surface below 45 degrees centigrade.

Still referring to FIG. 9, treatment distance L may be defined as extending from between points defined as being disposed beyond a proximal boundary M′ of the prostate and distal boundary of the prostate M″ by distances K′ and K″, respectively. For example, K′ and K″ may each equal one centimeter, and thus the massaging portion may be translated in back and forth fashion along L, treating the prostate and the area that extends on either side of the prostate by one centimeter. Distances K′ and K″ however may take any value.

In an alternative embodiment of the method of the invention, laser energy may be transmitted without translation of the probe over distance L. In this embodiment, the probe is adapted to transmit a wide beam of laser energy that covers the entire distance of tissue for which treatment is desired. In this alternative embodiment, probe body portion 100 is marked having a marking that, when the marking is at the level of the anus and the energy source is activated, laser energy is transmitted in a continuous or pulsatile fashion until the desired dosing is administered; i.e., the desired dosing parameter is met. The energy source is next inactivated and the probe removed.

Any of the embodiments directed to a method for treating a prostate may be facilitated by a group of novel device features. Such features include measurement markings upon the probe body portion. Such measurement marking provide the linear distance treated. Such features include markings on the probe body portion representing the range of rectum overlying the typical prostate and one cm marking or other equidistant markings between these markings, and a marking on the handle and or a handle shape that orients the energy, predominantly, toward the prostate. Ideally, the proximal and distal markings will be such that treatment between said markings results in a linear distance that extends one centimeter distal and one centimeter proximal to the prostate location above the rectum. A marking on the handle of the probe may facilitate orientation of the probe such that energy is transmitted predominantly toward the prostate. Auditory and or visual cues may be provided by the energy source user interface and or other hardware to facilitate the movement of the probe at the prescribed rate. Such cues shall rely upon the input of the probe diameter or power density information into the energy source and or an accelerometer contained within the probe or handle of the probe. Such cues may also rely upon surface temperature measurements taken by a temperature measuring mechanism within the probe that provides such temperature measurement to the energy source or accessory. In the preferred embodiment, the probe massaging portion has a greater outer dimension that the probe body portion such that a massage or milking effect is created. By way of example, the probe massaging portion may comprise a spherical shape, trapezoidal shape, conical shape or any other shape. Ideally but not necessarily, said probe massaging portion is disposed at or within one cm of the energy emitting area of the probe. A temperature sensor on the probe may provide feedback to the energy source.

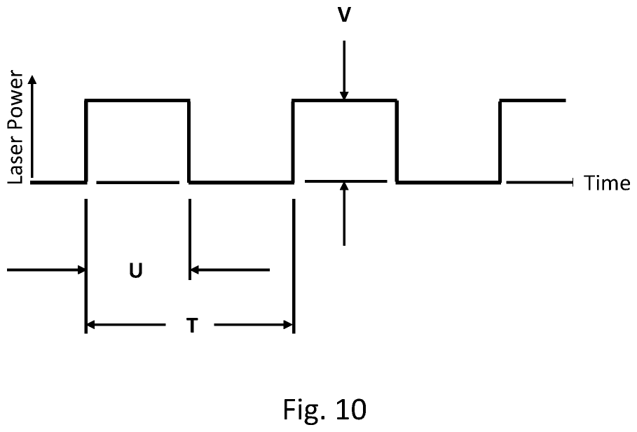

Referring now to FIG. 10, in any embodiment of the method of the invention using pulsatile laser energy, the pulse shape, meaning amplitude and width, frequency, and time as well as the time between pulses, i.e. duty cycle, may be adapted so as to maintain a probe surface temperature between 40 and 45 degrees centigrade. For example, referring to FIG. 10, an exemplary pulsatile waveform is depicted having a period T, a pulse width U, and an amplitude V. Any of these parameters may be changed in order to maintain a probe surface temperature between 40 and 45 degrees centigrade or to achieve a desired power or applied energy per linear centimeter, power or applied energy per square centimeter, or total applied energy. The duty cycle of the pulsatile waveform is defined as the pulse width U, measured in units of time, divided by the waveform period T, measured in units of time.

In any embodiment of the method of the invention, the desired treatment parameters may be any of the following, either alone or in any combination: 1) the administering of between 2000 4000 joules to the treatment area; 2) the administering of between 200-600 joules per linear centimeter to the treatment area; 3) the administering of between 21.068-149.208 joules/cm² to the treatment area; 4) or the administering of between 13.045-310.546 joules/cm² to the treatment area.

In any of the embodiments of the invention, the massaging portion and the light emitting portion may be fabricated from any material that is transmissive at near infrared wavelengths, such as silica glass, plastic, or any other material that is transmissive to laser energy over the defined near infrared wavelength range.

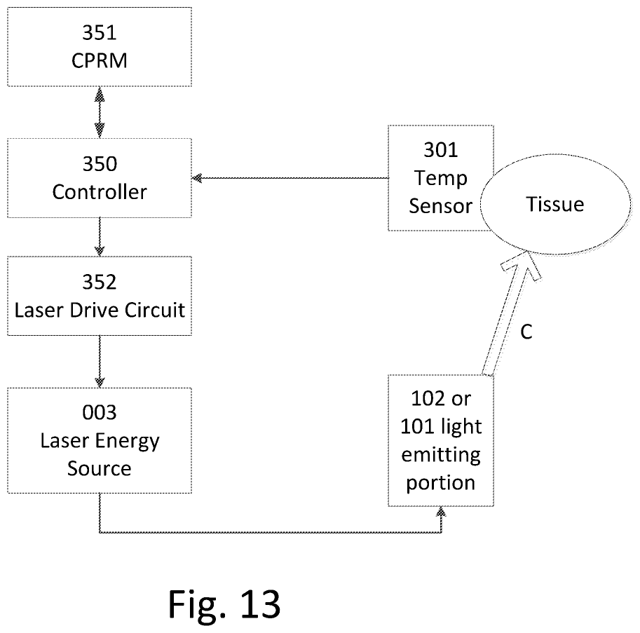

Regarding FIG. 11. The probe light emitting portion and massaging portion 101 may be the same structure, as depicted in FIGS. 3A, 3B, 4A, 4B, 5A and 5B. Likewise, an embodiment of the probe which further comprises a temperature sensor 301 and connecting wiring 300 is depicted in FIG. 11. Temperature sensor 301 may be connected to a controller 350 via wiring 300 as is further described in relation to the exemplary electrical block diagram of FIG. 13. Temperature sensor 301 may comprise any temperature sensor known in the art. For example, temperature sensor 301 may comprise a thermistor, semiconductor device, or any other electrical device that exhibits a change in at least one electrical characteristic proportional to, or in a known relationship to, changes in temperature. Temperature sensor 301 may comprises a thermally conductive element that is in physical contact with at least a portion of the tissue near the light emitting element of the probe, such that when the tissue changes temperature, the at least one electrical characteristic of the temperature probe also changes in a known relationship with the change in tissue temperature. Temperature probe 301 may be in communication with controller 350 (shown in FIG. 13). Controller 350 is adapted to measure the change in the at least one electrical characteristic of the temperature probe, and thereby is able to estimate the temperature of the tissue. Using this information, Controller 350 may then command the laser energy source 003 to either provide more or less power to achieve a particular tissue temperature, or to keep the tissue between a lower and upper limit of temperature. In an alternative embodiment, the temperature sensor may be comprised of an alexandrite fluorescence lifetime fiber optic thermometer in communication with controller 350. Probe body outer dimension B is shown for reference, as is probe longitudinal axis A and laser energy C being emitted massaging body 101a. In the embodiment shown in FIG. 11, the massaging portion and the light emitting portion are the same structure and may be fabricated from any material that is transmissive at near infrared wavelengths, such as silica glass, plastic, or any other material that is transmissive to laser energy over the defined near infrared wavelength range. Probe body 100 may comprise markings 202 at specific intervals, for example one centimeter spacing, to assist a surgeon with translating the probe over a predetermined linear distance.

Referring now to FIG. 12A, an embodiment of a probe of the invention in which the massaging portion 101a is separate from the light emitting portion 102a is depicted. In this embodiment the probe comprises a body portion 100, a light emitting portion 102a and a massaging portion 101a wherein light emitting portion is transmissive to near infrared laser energy; and wherein the probe is capable of being in optical communication with a source of laser energy 003 such that laser energy is transmissible from the laser source 003, through the body portion and into said light emitting portion 102a. The light emitting portion 102a is capable of emitting the laser energy C into tissue surrounding light emitting portion 102a when said laser source 003 is activated. The massaging portion 101a has an outer dimension D that is larger than the body portion outer dimension B. The probe configuration shown in FIG. 12A may be used to treat any body tissue but is also specifically adapted to emit laser energy C into vaginal tissue. Laser energy C may be emitted in a partially spherical radiation pattern as depicted in angles T-T and U-U in FIG. 6, wherein the spherical pattern is centered around an axis that is coaxial with the longitudinal axis of the probe A. Probe body 100 may comprise markings 202 at specific intervals, for example one centimeter spacing, to assist a surgeon with translating the probe over a predetermined linear distance. As described above, temperature sensor 301 may be in communication with controller 350 for measuring tissue temperature and controlling laser source 003 parameters, such as output power or duty cycle, or both, to achieve a desired tissue temperature range. In the embodiment shown in FIG. 12A, the light emitting portion 102a and the massaging portion 101a are separated by a distance Z as measured from the center of laser energy C emission from the light emitting portion 102a to the nearest surface of massaging portion 101a. An exemplary value for Z is one to two centimeters, but any other desired value for Z may be used. Massaging portion 101a is depicted as being spherically shaped in FIG. 12A, but the embodiments of the probe in which the massaging portion 101 is not the same structure as the light emitting portion may comprise any outer shape (e.g. spherical, conical, trapezoidal, or otherwise) for the massaging portion 101 or light emitting portions 102a.

Referring now to FIG. 12B, an embodiment of a probe of the invention in which the massaging portion 101a is separate from the light emitting portion 102a is depicted. In this embodiment the probe comprises a body portion 100, a light emitting portion 102a and a massaging portion 101a wherein light emitting portion is transmissive to near infrared laser energy; and wherein the probe is capable of being in optical communication with a source of laser energy 003 such that laser energy is transmissible from the laser source 003, through the body portion 100 and into the light emitting portion 102a. The light emitting portion 102a is capable of emitting the laser energy C into tissue surrounding light emitting portion 102a when said laser source 003 is activated. The massaging portion 101a has an outer dimension D that is larger than the body portion outer dimension B. The probe configuration shown in FIG. 12B may be used to treat any body tissue but is also specifically adapted to emit laser energy C into rectal tissue. Laser energy C may be emitted in a partially spherical radiation pattern as depicted in angles in FIGS. 8A and 8B, wherein the spherical pattern is centered around an axis N that is orthogonal to the longitudinal axis of the probe A. Probe body 100 may comprise markings 202 at specific intervals, for example one centimeter spacing, to assist a surgeon with translating the probe over a predetermined linear distance. As described above, temperature sensor 301 may be in communication with controller 350 for measuring tissue temperature and controlling laser source 003 parameters, such as output power or duty cycle, or both, to achieve a desired tissue temperature range. In the embodiment shown in FIG. 12B, the light emitting portion 102a and the massaging portion 101a are separated by a distance Z as measured from the center of laser energy C emission from the light emitting portion 102a to the nearest surface of massaging portion 101a. An exemplary value for Z is one to two centimeters. Massaging portion 101a is depicted as being spherically shaped in FIG. 12B, but the embodiments of the probe in which the massaging portion 101 is not the same structure as the light emitting portion may comprise any outer shape (e.g. spherical, conical, trapezoidal, or otherwise) for the massaging portion 101 or light emitting portions 102a.

Referring now to FIG. 13, the tissue temperature controlling feature of an embodiment of the invention is shown. A temperature measuring element 301 may be located on the probe as depicted, by way of example, in FIGS. 11, 12A and 12B. The temperature measuring element 301 may be any element for sensing a temperature and producing a signal proportional to the temperature, or which exhibits a change in electrical characteristic proportional to, or in a known relationship to, changes in temperature. Temperature sensing element 301 may comprise a thermally conductive element that is in physical contact with at least a portion of the tissue near the light emitting element of the probe, such that when the tissue changes temperature, the at least one electrical characteristic of the temperature probe also changes in a known relationship with the change in tissue temperature. Alternatively, temperature sensing element 301 may comprise a digital thermometer circuit that produces a digital output that is coded to represent the temperature of the measured tissue. Temperature probe 301 may be in communication with controller 350. Controller 350 is adapted to measure the change in the at least one electrical characteristic of the temperature probe, and thereby is able to estimate the temperature of the tissue. As an example, when controller 350 is a device that is adapted to perform functions by the execution of non-transitory computer readable instructions, such instructions may be stored in computer readable media (CRM) 351 that is in communication with controller 350. Controller 350 may read and execute the non transitory instructions stored in CRM 352 to perform the processing of the temperature signal from temperature sensing element 301 Using this information. Controller 350 may then command the laser energy source 003 to either provide more or less power to achieve a particular tissue temperature, or to keep the tissue between a lower and upper limit of temperature. This may be done by controller 350 directly controlling the output power or duty cycle of laser energy source 003, or by commanding an intervening laser drive circuit 352 is a controllable circuit adapted to receive a control signal from controller 350 and to control the output power or duty cycle of laser energy source. On activation, laser energy source 003 provides laser energy to the probe through an optically transmissive path, which may be, for example, an optical fiber that is transmissive at near infrared wavelengths. The laser energy is emitted through either the massaging portion 101 or light emitting portion 102 as depicted by arrow C into to the tissue surrounding the probe. Temperature sensing element 301 is in at least temporary physical contact with the tissue near to or surrounding the probe massaging portion. Temperature sensing element 301 provides a signal proportional to the temperature of the tissue as hereinbefore described, and controller 350 may then execute non-transitory computer readable instructions for reading the temperature of said tissue and controlling said laser drive circuit in order to adjust a laser source parameter selected from the group consisting of amplitude and duty cycle in order to maintain said tissue at a temperature between 40° and 45° centigrade, or any other temperature range desired or predetermined.

What is claimed is:

1. A method for treating pelvic pain, chronic prostatitis, or overactive bladder, comprising:
providing a source of laser energy;
providing a probe capable of emitting laser energy to vaginal tissue or rectal tissue, said probe in optical communication with said source of laser energy, said probe being characterized as having an outer surface;
inserting said probe into a vagina or rectum of a patient such that at least a portion of said probe outer surface is in physical contact with a portion of said vaginal or said rectal tissue;
activating said source of laser energy thereby causing laser energy to be emitted by said probe, wherein said laser energy illuminates said vaginal tissue in the case in which the probe is inserted into a vagina, or illuminates said rectal tissue in the case in which said probe is inserted into a rectum, creating a probe treatment surface area;
massaging said probe treatment surface area while illuminating said probe treatment surface area with said laser energy, said massaging performed by continuously translating said probe in a back and forth motion at a rate of motion along a longitudinal axis of said probe while said probe emits said laser energy, said rate of motion being between 2.5 and 60 millimeters per second, said translation being defined by a total distance of travel; and
administering a total dose of 200-600 joules per linear centimeter of tissue treated or 13.045-310.546 joules/cm2 at a power density between 0.09 W/cm2 and 6.0 W/cm².

2. The method of claim 1, wherein said laser energy is continuous

3. The method of claim 2,
wherein in the case in which said probe is inserted into a vagina, said probe is further defined as having a shape selected from the group consisting of a sphere of diameter between 1.5 and 4.0 cm that is capable of emitting said laser energy in a 120 degree to 360 degree spherical pattern along a longitudinal axis of said probe; and
wherein in the case in which the probe is inserted into a rectum, the probe is further defined as having a shape selected from the group consisting of a sphere a diameter between 1.5 and 4.0 cm that is capable of emitting said laser energy in a 60 degree to 180 degree spherical pattern about an axis that is transverse to a longitudinal axis of said probe, a trapezoid, and a cone; and
wherein, in the case in which said probe is inserted into a vagina, said step of continuously translating is performed without rotation of the probe within the vagina; and
wherein in the case in which said probe is inserted into a rectum, the probe is further defined as comprising markings to indicate a rotational orientation of the probe in which laser energy emitted from said probe is oriented predominantly towards the prostate.

4. The method of claim 2, wherein in the case in which the probe is inserted into a rectum, the total distance of travel extends one centimeter beyond the proximal and distal extents of the subject's prostate.

5. The method of claim 2, wherein said probe velocity during said continuous translation is between 10 mm/sec and 40 mm/sec.

6. The method of claim 2, wherein the power density to probe velocity ratio is between 0.0042 Wsec/cm³ and 1.8 Wsec/cm³.

7. The method of claim 1, wherein said laser energy is pulsatile.

8. The method of claim 7,
wherein in the case in which said probe is inserted into a vagina, said probe is further defined as having a shape selected from the group consisting of a sphere a diameter between 1.5 and 4.0 cm that is capable of emitting said laser energy in a 120 degree to 360 degree spherical pattern along a longitudinal axis of said probe, a trapezoid, and a cone; and
wherein in the case in which the probe is inserted into a rectum, the probe is further defined as having a shape selected from the group consisting of a sphere a diameter between 1.5 and 4.0 cm that is capable of emitting said laser energy in a 60 degree to 180 degree spherical pattern about an axis that is transverse to a longitudinal axis of said probe, a trapezoid, and a cone; and
wherein, in the case in which said probe is inserted into a vagina, said step of continuously translating is performed without rotation of the probe within the vagina; and
wherein in the case in which said probe is inserted into a rectum, the probe is further defined as comprising markings to indicate a rotational orientation of the probe in which laser energy emitted from said probe is oriented predominantly towards the prostate.

9. The method of claim 7, wherein in the case in which the probe is inserted into a rectum, the total distance of travel extends one centimeter beyond the proximal and distal extents of the subject's prostate.

10. The method of claim 7, wherein said probe velocity during said continuous translation is between 10 mm/sec and 40 mm/sec.

11. The method of claim 7, wherein the power density to probe velocity ratio is between 0.0042 Wsec/cm³ and 1.8 Wsec/cm³.

12. The method of claim 2, further comprising the step of providing markings on said probe to indicate the linear distance the probe has been translated during said continuous movement, said markings visible from outside said vagina when said probe is inserted into said vagina.

13. The method of claim 7, further comprising the step of providing markings on said probe to indicate the linear distance the probe is translated during said continuous movement, said markings visible from outside said vagina when said probe is inserted into said vagina.

14. The method of claim 2, further comprising the step of continuing said continuous translation until the applied energy has reached a predetermined dosage level, wherein said dosage level is calculated using the linear distance, and wherein said calculation results in joules per linear centimeter or joules per area.

15. The method of claim 7, further comprising the step of continuing said continuous translation until the applied energy has reached a predetermined dosage level, wherein said dosage level is calculated using the linear distance, and wherein said calculation results in joules per area.

16. The method of claim 12, wherein said markings are disposed on said probe connector at one centimeter intervals.

17. The method of claim 13, wherein said markings are disposed on said probe connector at one centimeter intervals.

18. The method of claim 2, further defining the step of providing a source of laser energy as that is operable to provide auditory or visual cues facilitating the continuous movement to be performed at a predetermined rate.

19. The method of claim 7, further defining the step of providing a source of laser energy as that is operable to provide auditory or visual cues facilitating the continuous movement to be performed at a predetermined rate.

20. The method of claim 14, further including the step of providing input to said auditory or visual cues, said input selected from the group consisting of providing probe diameter and or power density, accelerometer information, probe treatment surface area, and temperature information; and further comprising the step of processing said input to determine the timing of said auditory or visual cues.

21. A method for treating pelvic pain, chronic prostatitis, or overactive bladder, comprising:
providing a source of laser energy;
providing a probe capable of emitting laser energy to vaginal tissue or rectal tissue, said probe in optical communication with said source of laser energy, said probe being characterized as having an outer surface;
inserting said probe into a vagina or rectum of a patient such that at least a portion of said probe outer surface is in physical contact with a portion of said vaginal or said rectal tissue;
activating said source of laser energy thereby causing laser energy to be emitted by said probe, wherein said laser energy illuminates said vaginal tissue in the case in which the probe is inserted into a vagina, or illuminates said rectal tissue in the case in which said probe is inserted into a rectum, creating a probe treatment surface area;
massaging said probe treatment surface area while illuminating said probe treatment surface area with said laser energy, said massaging performed by continuously translating said probe in a back and forth motion at a rate of motion along a longitudinal axis of said probe while said probe emits said laser energy, said rate of motion being between 2.5 and 60 millimeters per second, said translation being defined by a total distance of travel; and
administering a total dose of 200-600 joules per linear centimeter of tissue treated or 13.045-310.546 joules/cm2 at a power density between 0.09 W/cm2 and 6.0 W/cm2.

22. The method of claim 21, wherein said laser energy is continuous.

23. The method of claim 22,
wherein in the case in which said probe is inserted into a vagina, said probe is further defined as having a shape selected from the group consisting of a sphere a diameter between 1.5 and 4.0 cm that is capable of emitting said laser energy in a 120 degree to 360 degree spherical pattern along a longitudinal axis of said probe, a trapezoid, and a cone; and
wherein in the case in which the probe is inserted into a rectum, the probe is further defined as having a shape selected from the group consisting of a sphere a diameter between 1.5 and 4.0 cm that is capable of emitting said laser energy in a 60 degree to 180 degree spherical pattern about an axis that is transverse to a longitudinal axis of said probe, a trapezoid, and a cone; and
wherein, in the case in which said probe is inserted into a vagina, said step of continuously translating is performed without rotation of the probe within the vagina; and
wherein in the case in which said probe is inserted into a rectum, the probe is further defined as comprising markings to indicate a rotational orientation of the probe in which laser energy emitted from said probe is oriented predominantly towards the prostate.

24. The method of claim 22, wherein in the case in which the probe is inserted into a rectum, the total distance of travel extends one centimeter beyond the proximal and distal extents of the subject's prostate.

25. The method of claim 22, wherein said probe velocity during said continuous translation is further defined as between 10 mm/sec and 40 mm/sec.

26. The method of claim 17, wherein the power density to probe velocity ratio is between 0.0042 Wsec/cm³ and 1.8 Wsec/cm³.

27. The method of claim 21, wherein said laser energy is pulsatile.

28. The method of claim 27,
wherein in the case in which said probe is inserted into a vagina, said probe is further defined as having a shape selected from the group consisting of a sphere a diameter between 1.5 and 4.0 cm that is capable of emitting said laser energy in a 120 degree to 360 degree spherical pattern along a longitudinal axis of said probe, a trapezoid, and a cone; and
wherein in the case in which the probe is inserted into a rectum, the probe is further defined as having a shape selected from the group consisting of a sphere a diameter between 1.5 and 4.0 cm that is capable of emitting said laser energy in a 60 degree to 180 degree spherical pattern about an axis that is transverse to a longitudinal axis of said probe, a trapezoid, and a cone; and
wherein, in the case in which said probe is inserted into a vagina, said step of continuously translating is performed without rotation of the probe within the vagina; and
wherein in the case in which said probe is inserted into a rectum, the probe is further defined as comprising markings to indicate a rotational orientation of the probe in which laser energy emitted from said probe is oriented predominantly towards the prostate.

29. The method of claim 27, wherein in the case in which the probe is inserted into a rectum, the total distance of travel extends one centimeter beyond the proximal and distal extents of the subject's prostate.

30. The method of claim 27, wherein said probe velocity during said continuous translation is further defined between 10 mm/sec and 40 mm/sec

31. The method of claim 22, wherein the power density to probe velocity ratio is between 0.0042 Wsec/cm³ and 1.8 Wsec/cm³.

32. The method of claim 22 and, further comprising the step of providing markings on said probe to indicate the linear distance the probe has been translated during said continuous movement, said markings visible from outside said vagina when said probe is inserted into said vagina.

33. The method of claim 27, further comprising the step of providing markings on said probe to indicate the linear distance the probe has been translated during said continuous movement, said markings visible from outside said vagina when said probe is inserted into said vagina.

34. The method of claim 32, wherein said markings are disposed on said probe connector at one centimeter intervals.

35. The method of claim 33, wherein said markings are disposed on said probe connector at one centimeter intervals.

36. The method of claim 22, further defining the step of providing a source of laser energy as that is operable to provide auditory or visual cues facilitating the continuous movement to be performed at a predetermined rate.

37. The method of claim 36, further including the step of providing input to said auditory or visual cues, said input selected from the group consisting of probe diameter or power density information, accelerometer information, probe treatment surface area, and temperature information; and further comprising the step of processing said input to determine the timing of said auditory or visual cues.

38. The method of claim 27, further defining the step of providing a source of laser energy as providing a source of laser energy that is operable to provide auditory or visual cues facilitating the continuous movement to be performed at a predetermined rate.

39. The method of claim 38, further including the step of providing input to said auditory or visual cues, said input selected from the group consisting of probe diameter or power density information, accelerometer information, probe treatment surface area, and temperature information; and further comprising the step of processing said input to determine the timing of said auditory or visual cues.

40. The method of claim 22 wherein the linear distance is input to facilitate preferred dosing

41. The method of claim 27 wherein the linear distance is input to facilitate preferred dosing

42. A method for treating pelvic pain, chronic prostatitis, or overactive bladder, comprising:
providing a source of laser energy;
providing a probe capable of emitting laser energy to vaginal tissue or rectal tissue, said probe in optical communication with said source of laser energy, said probe being characterized as having an outer surface;
inserting said probe into a vagina or rectum of a patient such that at least a portion of said probe outer surface is in physical contact with a portion of said vaginal or said rectal tissue;
activating said source of laser energy thereby causing laser energy to be emitted by said probe, wherein said laser energy illuminates said vaginal tissue in the case in which the probe is inserted into a vagina, or illuminates said rectal tissue in the case in which said probe is inserted into a rectum, creating a probe treatment surface area;
massaging said probe treatment surface area while illuminating said probe treatment surface area with said laser energy, said massaging performed by continuously translating said probe in a back and forth motion at a rate of motion along a longitudinal axis of said probe while said probe emits said laser energy, said rate of motion being between 2.5 and 60 millimeters per second, said translation being defined by a total distance of travel; and
administering between 200 J/cm² and 600 J/cm to the vaginal tissue or rectal tissue.

43. The method of claim 31, wherein said laser energy is continuous.

44. The method of claim 32,
wherein in the case in which said probe is inserted into a vagina, said probe is further defined as having a shape selected from the group consisting of a sphere a diameter between 1.5 and 4.0 cm that is capable of emitting said laser energy in a 120 degree to 360 degree spherical pattern along a longitudinal axis of said probe, a trapezoid, and a cone; and
wherein in the case in which the probe is inserted into a rectum, the probe is further defined as having a shape selected from the group consisting of a sphere a diameter between 1.5 and 4.0 cm that is capable of emitting said laser energy in a 60 degree to 180 degree spherical pattern about an axis that is transverse to a longitudinal axis of said probe, a trapezoid, and a cone; and
wherein, in the case in which said probe is inserted into a vagina, said step of continuously translating is performed without rotation of the probe within the vagina; and
wherein in the case in which said probe is inserted into a rectum, the probe is further defined as comprising markings to indicate a rotational orientation of the probe in which laser energy emitted from said probe is oriented predominantly towards the prostate.

45. The method of claim 32, wherein in the case in which the probe is inserted into a rectum, the total distance of travel extends one centimeter beyond the proximal and distal extents of the subject's prostate.

46. The method of claim 32, wherein said probe velocity during said continuous translation is further defined between 10 mm/sec and 40 mm/sec.

47. The method of claim 32, wherein the power density to probe velocity ratio is between 0.0042 Wsec/cm³ and 1.8 Wsec/cm³.

48. The method of claim 31, wherein said laser energy is pulsatile.

49. The method of claim 37,
wherein in the case in which said probe is inserted into a vagina, said probe is further defined as having a shape selected from the group consisting of a sphere a diameter between 1.5 and 4.0 cm that is capable of emitting said laser energy in a 120 degree to 360 degree spherical pattern along a longitudinal axis of said probe, a trapezoid, and a cone; and
wherein in the case in which the probe is inserted into a rectum, the probe is further defined as having a shape selected from the group consisting of a sphere a diameter between 1.5 and 4.0 cm that is capable of emitting said laser energy in a 60 degree to 180 degree spherical pattern about an axis that is transverse to a longitudinal axis of said probe, a trapezoid, and a cone; and
wherein in the case in which said probe is inserted into a vagina, said step of continuously translating is performed without rotation of the probe within the vagina; and
wherein in the case in which said probe is inserted into a rectum, the probe is further defined as comprising markings to indicate a rotational orientation of the probe in which laser energy emitted from said probe is oriented predominantly towards the prostate.

50. The method of claim 37, wherein in the case in which the probe is inserted into a rectum, the total distance of travel extends one centimeter beyond the proximal and distal extents of the subject's prostate.

51. The method of claim 37, wherein said probe velocity during said continuous translation is further defined between 10 mm/sec and 40 mm/sec.

52. The method of claim 37, wherein the power density to probe velocity ratio is between 0.0042 Wsec/cm³ and 1.8 Wsec/cm³.

53. The method of claim 32, further comprising the step of providing markings on said probe to indicate the linear distance the probe has been translated during said continuous movement, said markings visible from outside said vagina when said probe is inserted into said vagina.

54. The method of claim 42, wherein said markings are disposed on said probe connector at one centimeter intervals.

55. The method of claim 32, further defining the step of providing a source of laser energy as providing a source of laser energy that is operable to provide auditory or visual cues facilitating the continuous movement to be performed at a predetermined rate.

56. The method of claim 44, further including the step of providing input to said auditory or visual cues, said input selected from the group consisting of providing probe diameter or surface area information, accelerometer information, probe treatment surface area, and temperature information; and further comprising the step of processing said input to determine the timing of providing said auditory or visual cues.

57. A method for treating pelvic pain, chronic prostatitis, or overactive bladder, comprising:
providing a source of pulsed laser energy having a duty cycle, said duty cycle defined as having a period and a pulse width that is less than said period, and a amplitude;
providing a probe capable of emitting laser energy to vaginal tissue, said probe in optical communication with said source of laser energy, said probe having a spherical, trapezoidal, or conical shaped laser energy emitting distal end being capable of emitting said laser energy in a 120 degree to 360 degree spherical pattern along a longitudinal axis of said probe;
establishing a total laser probe travel distance defined as the extent of desired laser probe travel within the vagina between the most proximal and most distal points of treatment;
inserting said probe into a vagina of a patient such that at said probe is located along said total laser probe travel distance least a portion of said probe is located and is in physical contact with a portion of said vaginal tissue;
activating said source of laser energy, causing laser energy to be emitted by said probe, wherein said laser energy illuminates said vaginal tissue in the vicinity of said probe;
administering near infrared light at a power density of between 0.09 W/cm² and 6.0 W/cm² to the vaginal tissue;
deactivating said laser energy source after the total laser energy delivered to said vaginal tissue is between 21.068 and 149.208 joules/cm²; and
advancing or withdrawing said probe by one centimeter, reactivating said laser energy source until the total laser energy delivered to said vaginal tissue is between 21.068 and 149.208 joules/cm² and repeating at increments of one centimeter along the total laser probe travel distance until said probe has traversed the entire length of the total laser probe travel distance.

58. The method of claim 46, further comprising the step of providing markings on said probe to indicate the linear distance the probe has been translated or the distance of insertion of said probe into said vagina, said markings visible when said probe is inserted into said vagina.

59. The method of claim 46, further comprising the step of adjusting either the pulse width or amplitude of said duty cycle to maintain a vaginal tissue surface treatment temperature of between 40 degrees centigrade and 45 degrees centigrade.

60. A method for treating pelvic pain, chronic prostatitis, or overactive bladder, comprising:
providing a source of pulsed laser energy having a duty cycle;
providing a probe capable of emitting pulsed laser energy to rectal tissue, said probe in optical communication with said source of laser energy, said probe having a spherical, trapezoidal, or conical shaped laser energy emitting distal end being capable of emitting said laser energy in a 60 degree to 180 degree spherical pattern predominantly along an axis that is transverse to a longitudinal axis of the probe;
establishing a total laser probe travel distance defined as the extent of desired laser probe travel within the rectum, the most proximal and most distal points of total laser probe travel being defined as one centimeter beyond the proximal and distal margins of the prostate;
inserting said probe into a rectum of a patient such that at said probe is located along said total laser probe travel distance and at least a portion of said probe is located and is in physical contact with a portion of said rectal tissue;
activating said source of laser energy, causing pulsed laser energy to be emitted by said probe, wherein said pulsed laser energy illuminates said rectal tissue overlying said prostate;
administering near infrared light at a power density of between 0.09 W/cm² and 6.0 W/cm² to said rectal tissue;
deactivating said laser energy source after the total laser energy delivered to said rectal tissue is between 21.068 and 149.208 joules/cm²; and
advancing or withdrawing said probe by one centimeter, reactivating said laser energy source until the total laser energy delivered to said rectal tissue is between 21.068 and 149.208 joules/cm² and repeating at increments of one centimeter along the total laser probe travel distance until said probe has traversed the entire length of the total laser probe travel distance.

61. The method of claim 49, wherein said spherical shaped laser energy emitting distal end has a diameter between 1.5 and 4.0 cm.

62. The method of claim 49, wherein said preferred energy dose is administered whilst moving said probe between a first mark on said probe and a second mark on said probe that are visualized at anus, such that said first mark on said probe is three or less centimeters from a distal end of said probe, and said second mark on said probe that is 16 or less centimeters from said distal end of said probe.

63. The method of claim 49, further comprising the step of providing markings on said probe connector to indicate the linear distance the probe has been translated or the distance of insertion of said probe into said rectum, said markings visible when said probe is inserted into said rectum.

64. The method of claim 49, further comprising the step of adjusting said pulsed laser energy duty cycle to maintain a rectal tissue surface treatment temperature below 45 degrees centigrade.

65. The method of claim 49, wherein said probe connector is further defined as having markings at one centimeter spacing.

66. The method of claim 49, wherein said probe or probe handle is defined as having a laser energy emitting marking or shape indicating the orientation of said pulsed laser energy, and further comprising the step of aligning the laser energy emitting marking or shape such that said pulsed laser energy is emitted toward said prostate.

67. The method of claim 60, further comprising the step of repeating the step of deactivating, advancing or withdrawing, and reactivating until the applied energy has reached a predetermined dosage level wherein said dosage level is calculated using the linear distance, and wherein said calculation results in joules per linear centimeter, joules per area.

68. A device for treating pelvic pain, chronic prostatitis, or overactive bladder, comprising:
a probe comprising a body portion and a massaging portion;
wherein each of said probe and massaging portion have an outer dimension and are transmissive to said laser energy; and
wherein said probe is capable of being in optical communication with a source of laser energy such that laser energy is transmissible from the laser source, through the body portion and into the massaging portion; and
wherein said massaging portion is capable of emitting said laser energy into tissue surrounding said massaging portion when said laser source is activated;
and wherein said massaging portion has an outer dimension that is larger than said body portion.

69. The device of claim 68, wherein said laser energy is near infrared in wavelength.

70. The device of claim 68, wherein said body portion is between 0.5 cm and 4.0 cm in length.

71. The device of claim 68, wherein said massaging portion outer dimension is at least 0.2 cm greater than said body portion outer dimension.

72. The device of claim 69, wherein said massaging portion outer dimension is at least 0.2 cm greater than said body portion outer dimension.

73. The device of claim 68, wherein said body portion is marked in periodic increments.

74. The device of claim 73, wherein said markings are 1 cm apart.

75. The device of claim 68, wherein said massaging portion is adapted to emit said laser energy in a partially spherical pattern covering at least 120°, said spherical pattern centered about an axis that is coaxial with a longitudinal axis of said probe.

76. The device of claim 68, wherein said massaging portion is adapted to emit said laser energy in a spherical pattern covering up to 360°, said spherical pattern centered about an axis that is coaxial with a longitudinal axis of said probe.

77. The device of claim 68, wherein said massaging portion is spherically shaped.

78. The device of claim 68, wherein said massaging portion is conically shaped.

79. The device of claim 68, wherein said massaging portion is trapezoidally shaped.

80. The device of claim 68 further comprising:
a temperature measuring element located on said probe, said temperature measuring element producing a signal proportional to a temperature of said tissue;
a controller; and
a laser drive circuit in communication with said laser source, said laser drive circuit capable of activating and deactivating said laser source, said laser drive circuit in communication with said controller;
wherein said temperature measuring element is in at least temporary physical contact with said tissue, and wherein said temperature measuring element is in communication with said controller;
wherein said controller is capable of executing non-transitory computer readable instructions for reading the temperature of said tissue and controlling said laser drive circuit in order to adjust a laser source parameter selected from the group consisting of amplitude and duty cycle in order to maintain said tissue at a temperature between 40° and 45° centigrade.

81. The device of claim 68, wherein said massaging is adapted to emit said laser energy in a direction orthogonal to a longitudinal axis of the probe, said laser energy emitted in a partially spherical pattern covering up to 180°.

82. The device of claim 68, wherein said massaging is adapted to emit said laser energy in a direction orthogonal to a longitudinal axis of the probe, said laser energy emitted in a partially spherical pattern covering at least 30°.

83. A device for treating pelvic pain, chronic prostatitis, or overactive bladder, comprising:
a probe comprising a body portion, a light emitting portion and a massaging portion;
wherein each of said probe and massaging portion have an outer dimension and are transmissive to said laser energy; and
wherein said probe is capable of being in optical communication with a source of laser energy such that laser energy is transmissible from the laser source, through said body portion and into said light emitting portion; and
wherein said light emitting portion is capable of emitting said laser energy into tissue surrounding said light emitting portion when said laser source is activated;
and wherein said massaging portion has an outer dimension that is larger than said body portion.

84. The device of claim 83, wherein said laser energy is near infrared in wavelength.

85. The device of claim 83, wherein said body portion is between 0.5 cm and 4.0 cm in length.

86. The device of claim 83, wherein said massaging portion outer dimension is at least 0.2 cm greater than said body portion outer dimension.

87. The device of claim 84, wherein said massaging portion outer dimension is at least 0.2 cm greater than said body portion outer dimension.

88. The device of claim 83, wherein said body portion is marked in periodic increments.

89. The device of claim 88, wherein said markings are 1 cm apart.

90. The device of claim 83, wherein said massaging portion is adapted to emit said laser energy in a partially spherical pattern covering at least 120°, said spherical pattern centered about an axis that is coaxial with a longitudinal axis of said probe.

91. The device of claim 83, wherein said massaging portion is adapted to emit said laser energy in a spherical pattern covering up to 360°, said spherical pattern centered about an axis that is coaxial with a longitudinal axis of said probe.

92. The device of claim 83, wherein said massaging portion is spherically shaped.

93. The device of claim 83, wherein said massaging portion is conically shaped.

94. The device of claim 83, wherein said massaging portion is trapezoidally shaped.

95. The device of claim 83 further comprising:
a temperature measuring element located on said probe, said temperature measuring element producing a signal proportional to a temperature of said tissue;
a controller; and
a laser drive circuit in communication with said laser source, said laser drive circuit capable of activating and deactivating said laser source, said laser drive circuit in communication with said controller;
wherein said temperature measuring element is in at least temporary physical contact with said tissue, and wherein said temperature measuring element is in communication with said controller;
wherein said controller is capable of executing non-transitory computer readable instructions for reading the temperature of said tissue and controlling said laser drive circuit in order to adjust a laser source parameter selected from the group consisting of amplitude and duty cycle in order to maintain said tissue at a temperature between 40° and 45° centigrade.

96. The device of claim 83, wherein said massaging is adapted to emit said laser energy in a direction orthogonal to a longitudinal axis of the probe, said laser energy emitted in a partially spherical pattern covering up to 180°.

97. The device of claim 96, wherein said body portion is marked in periodic increments, with said markings separated by a distance that is equal to the distance corresponding to the length of prostate overlying the rectum, or length slightly longer than the length of the prostate overlying the rectum, or a length equal to the length of the prostate to be treated.

98. The device of claim 97 with the markings on probe located such that they are exterior to the rectum during treatment.

99. The device of claim 98 with a marking on the handle or handle shape that facilitates proper orientation of the probe such that light energy emitted from the probe is illuminates the prostate.

100. The device of claim 83, wherein said massaging is adapted to emit said laser energy in a direction orthogonal to a longitudinal axis of the probe, said laser energy is emitted in a partially spherical pattern covering at least 30°.

101. The device of claim 83, wherein said light emitting portion and said massaging portion are located on said probe such that their facing surfaces are separated by no more than 2 cm.
